(19) United States
(12) Patent Application Publication
(10) Pub. No.: US 2015/0038376 A1
(43) Pub. Date: Feb. 5, 2015

(54) THYROID CANCER BIOMARKER

(71) Applicant: QIAGEN SCIENCES LLC, Germantown, MD (US)

(72) Inventors: Song Tian, Germantown, MD (US); Xiao Zeng, Frederick, MD (US); John Dicarlo, Bethesda, MD (US); Jiaye Yu, Durham, NC (US); Thomas J. Fahey, Larchmont, NY (US); Vikram Devgan, Frederick, MD (US); George J. Quellhorst, Rockville, MD (US); Raymond K. Blanchard, Frederick, MD (US)

(21) Appl. No.: 14/384,902

(22) PCT Filed: Mar. 15, 2013
(86) PCT No.: PCT/US13/32116

§ 371 Date: Sep. 12, 2014

Related U.S. Application Data
(60) Provisional application No. 61/611,179, filed on Mar. 15, 2012.

(51) Int. Cl. C12Q 1/68

(52) U.S. Cl. CPC C12Q 1/686
USPC 506/17

(57) ABSTRACT

The methods provided herein use microarray data for feature selection and then use selected targets to generate industry standard qPCR arrays with new clinical sample assay data so order to build a classification model. This multi-step method overcomes the disadvantages of traditional biomarker identification.

### BACKGROUND OF THE INVENTION

1. Sequence Listing

The instant application contains a Sequence Listing which has been submitted in ASCII format via EFS-Web and is hereby incorporated by reference in its entirety. Said ASCII copy, created on Mar. 5, 2013, is named 0051-0096-WOI_SL.txt and is 5,019 bytes in size.

2. Field of the invention

The methods provided herein use microarray data for feature selection and then use selected targets to generate industry standard quantitative real-time (qPCR) arrays with new clinical sample assay data in order to build a classification model. This multi-step method overcomes the disadvantages of traditional biomarker identification.

3. Background of the Invention

There are challenges in clinical classification of thyroid nodules using traditional methods. These challenges affect clinical decision making and lead to performance of unnecessary operations. While some researchers have explored the use of novel molecular classification methods to overcome these challenges, these efforts are still far from implementation in clinical settings.

Thyroid nodules are common in most populations. For example, it was estimated that 44,670 new patients would be identified in the United States in 2010. Often invasive diagnostic methods are necessary for accurate diagnosis of nodule types in patients. Fine-needle aspiration biopsy (FNAB) provides the most important diagnostic tool, since it was introduced. In 1970s, yet 20-30% of FNAB cytology results am still indeterminate. Although indeterminate, suspicious or non-diagnostic FNABs can be-repeated, these are only helpful for a small percentage of patients and require additional costs and invasive procedures.

Many researchers have attempted to develop additional, diagnostic assays and biomarkers to improve diagnostic accuracy. For example, fine needle aspiration cytology (FNAC) has its value in better accuracy but the limitation is clear especially in Follicular Thyroid Carcinoma (FTC). Immunohistochemical biomarkers such as Hector Battifora mesothelial cell 1 (HBME-1), high molecular weight Cytokeratin 19 (CK19) and Galectin-3 have been shown to have thyroid carcinoma, related expression, but their expression is highly variable in sensitivity and specificity. Other efforts, such as studies using somatic mutations and/or gene rearrangements m malignant thyroid cells, have made limited progress. Farther research has focused on Rearranged in Transformation/Papillary Thyroid Carcinomas (RET/PTC) in which rearrangements and mutations of the BRAF and RAS genes have been found to increase the accuracy of diagnosis, prognosis and validation studies. Lastly, microarray gene profiling has been shown to benefit classification of benign nodules and malignant tumors. However, most of these studies are only focused on simple microarray analysis and validation to identify genes that were differentially expressed between the benign and malignant groups. It is clear that a more robust assay and more delicate analysis with biomformatics models will better fit the challenge of tumor heterogeneity and the complexity of clinical samples, especially for thyroid cancer.

Microarray-based assays, however, have some inherent, drawbacks. They are sensitive to sample quality, which often presents challenges in a clinical setting. Microarray-based technologies also require increased sample preparation time and complicated data analysis procedures.

Traditionally, microarrays were directly used for biomarker signature generation. However, direct use of microarrays resulted in many challenges in clinical settings, and although some important targets were observed, no consensus on how to translate observations made through microarray experiments into user-friendly clinical tests developed. An additional drawback to the traditional direct use of microarrays was the standardization between different microarray platforms. Multiple microarray platforms exist, each of which use distinct sets of genes and employ different hybridization and signal-detection methods. For example, some microarrays contain cBNAs of variable lengths while others contain small oligonucleotide sequences. The use of different microarray platforms necessitates additional normalization and conversion work between platforms, making results less consistent and increasing the risk of errors.

Researchers have used traditional discovery cluster analysis such as unsupervised hierarchical clustering and 2 group k-mean clustering for target identification and final classification for thyroid cancer identification. Besides the well designed multiple model-based feature selection and qPCR array optimization, provided herein is a new training sample set for supervised machine learning which is then used in a well-accepted classification method—Random forest for the final malignant thyroid nodule identification.

Traditionally, the usage of discovery tools for classification limited their potential use for clinical diagnosis. Marschall Stevens Range in his book “Principles of molecular medicine” states, “[u]nsupervised methods of analysis, including principal component analysis, hierarchical clustering, k-means clustering, and self-organizing maps, can be used as tools for class discovery.” Moreover, “[u]nsupervised approaches to determine differences in gene expression profiles among disease states have limitations that can be circumvented by the use of supervised learning methods.” The methods provided herein use supervised machine learning methods for the classification of malignant thyroid nodules and benign nodules and avoid the problems and limitations of previous methods.

### SUMMARY OF THE INVENTION

In embodiments, quantitative real-time polymerase chain reaction (qPCR) arrays mare provided. Suitably, the arrays comprise one or more thyroid nodule malignancy classification biomarkers selected from NPC2, S100A11, SDC4, CD53, MET, GCSH, and CHI3L1: one or more reference genes selected from TBP, RPL13A, RPS13, HSP90A81 and YWHAZ; and a companion classifying algorithm for producing a single malignancy score and a scalable cut-off threshold.

Suitably, The arrays comprise 3 or more of the thyroid nodule malignancy classification biomarkers and 3 or more of the reference genes, more suitably the arrays comprise 5 or more of the thyroid nodule malignancy classification biomarkers and 4 or more of the reference genes.

In embodiments, the arrays comprise the thyroid nodule malignancy classification biomarkers NP2, S100A11, SDC4, CD53, MET, GCSH, and CH13L1 and the reference genes TBP, RPL13A, RPS13, HSP90A81 and YWHAZ.

Exemplary replacement genes for use in the arrays are described herein, as are exemplary mathematic models for use in the algorithms

### BRIEF DESCRIPTION OF THE DRAWINGS

FIG. 1 shows an example of a development roadmap for preparing a biomarker PCR array as described herein.

FIG. 2 shows a qPCR array development process as described herein.

FIG. 3 shows a workflow from sample to biomarker signature panel using a qPCR array system as described herein.

FIGS. 4A-4D show the development of a thyroid malignancy qPCR array, as described herein.

FIG. 5 shows the results of a thyroid malignancy signature.

FIG. 6A shows the sequence for Homo Sapiens TATA box binding protein (TBP), transcript variant 2, mRNA (SEQ ID NO: 1).

FIG. 6B shows the sequence for Homo Sapiens TATA box binding protein (TBP), transcript variant 1, mRNA (SEQ ID NO:2).

FIG. 7A shows the sequence for Homo sapiens Niemann-pick disease, type C2 (NPC2), mRNA (SEQ ID NO: 3).

FIG. 7B shows the sequence for Homo sapiens S100 calcium binding protein A11 (S100A11), mRNA (SEQ ID NO:4).

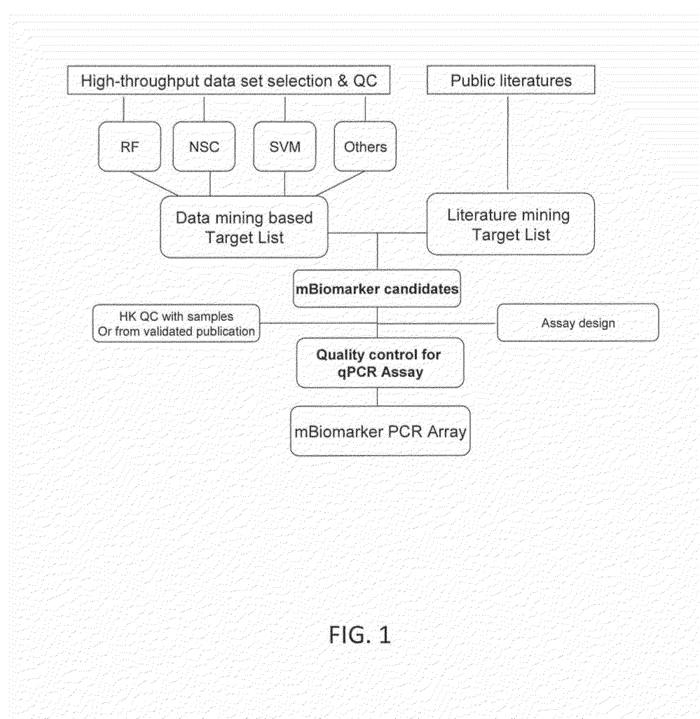

### DETAILED DESCRIPTION OF PREFERRED EMBODIMENTS

It should be appreciated that the particular implementations shown and described herein ate examples and are not intended to otherwise limit the scope of the application in any way.

The published patents, patent applications, websites, company names and scientific literature referred to herein are hereby incorporated by reference in their entireties to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries: above and below the numerical values set forth. In general, the term “about” is used herein to modify a numerical value above and below the stated value by a variance of 20%.

Technical, and scientific terms used herein have the meaning commonly understood by one of skill in the art to which the present application pertains, unless otherwise defined. Reference is made herein to various methodologies and materials known to those of ordinary skill in the art.

Development of biomarker qPCR Array

In embodiments, methods of preparing a biomarker quantitative real-time polymerase chain reaction (qPCR) array are provided. Suitably, such methods comprise selecting one or more high-throughput feature expression data sets, normalizing the feature expression, data sets, analyzing the data sets by one or more mathematical models to yield final candidate features, and generating the biomarker qPCR array comprising the final candidate features.

As used herein, a “biomarker” refers to a measurable characteristic that provides information on presence and/or severity of a disease or compromised state in a patient; the relationship tea biological pathway; a pharmacodynamic relationship or output; a companion diagnostic; a particular species; or a quality of a biological sample. Examples of biomarkers include genes, proteins, peptides, antibodies, cells, gene products, enzymes, hormones, etc.

As used herein a “feature” refers to a genes, portions of genes or other genomic information. Suitably, a feature- refers to a gene that is utilized to prepare an array as described herein.

In embodiments, the one or more high-throughput feature expression, data sets (including microarray data, sets, as well as other sequencing data sets including next generation sequencing platforms) are selected based on one or more of clinical utility (e.g. disease specific biomarkers), research interest (e.g., biological pathway-specific biomarkers), drug response (e.g., pharmacodynamic biomarkers or companion diagnostic biomarkers), species and quality.

In embodiments, the analyzing comprises analysis of the data sets with one or more mathematical models including but not limited to. Random forest (RF) modeling, support vector machine (SVM) modeling and nearest shrunken centroid (NSC) modeling. Additional models known in the art can also be utilized in the methods described herein, including for example, various genetic algorithms, decision tress and Naive Bayes modeling.

Methods of conducting such modeling are well known in the art, and described for example, RF models are described in Touw et al., “Data mining in the Life Sciences with Random Forest: a walk in the park or lost in the jungle?,” Briefings in Bioinformatics, May 26, 2012, Kursa and Rudnicki, “The All Relevant Feature Selection using Random Forest,” Cornell University Library, arXiv: 1106.5112, Jun. 25, 2011, Genuer et al., “Variable Selection using Random Forests,” Paper Submitted to Pattern Recognition Letters, Mar. 17, 2010, Ostroff et al., “Early Detection of Malignant Pleural Mesothelioma in Asbestos-Exposed Individuals with a Noninvasive Proteomics-Based Surveillance Tool” PLOS ONE 7:e46091 (Oct. 2012), Chen et al., “Development and Validation of a qRT-PCR Classifier for Lung Cancer Prognosis,” J. Thorac. Onocl. 6:1481-1487 (September 2011); NSC models are described in Klassen and Kim, “Nearest Shrunken Centroid as Feature Selection of Microarray Data, available at http://www.research.gate.net/, Tibshirani et al., “Diagnosis of multiple cancer types by shrunken centroids of gene expression,” Proc. Natl. Acad. Sci. 99:6567-6572 (May 14, 2002); and SVM models are described in Yonsef et al., “Classification and biomarker identification using gene network molecules and support vector machines,” BMC Bioinformatics 10:337 (2009), and Brank, J., “Feature Selection Using Linear Support Vector Machines,” Microsoft Research Technical Report, MSR-TR-2002-63 (Jun. 12, 2002) (the disclosure of each of which is incorporated by reference herein in their entireties, specifically for the disclosure of the models described herein and their implementation). In embodiments, the analysis comprises use of two, or more suitably, all three of these models on the data to generate the combined feature set and the final qPCR array.

Suitably, the analyzing comprises combining discriminative features from one or more of the mathematical models based on a desired classification implied by the data sets. That is, depending on the desired analysis (i.e., clinical outcome, research interest, etc), features that discriminate between one biomarker and another are selected. For example, genes that are present in a disease state are selected over genes that are not indicative of the disease state or other characteristic.

As described herein, the analysis can further comprise literature mining to yield the final candidate matures. This allows for the addition of further information to clarify and define the desired candidate features.

Suitably, the methods further comprise selecting one or more control data sets for inclusion of control features in the biomarker qPCR array. As described herein, it is the selection of these control features (i.e., features that do not demonstrate a change in a biomarker characteristic) that provides one of the unique features of the methods and arrays provided herein, so as to produce the most useful array information.

Also provided are qPCR arrays prepared by the methods described herein. In suitable embodiments, each defined location in an array corresponds to a biological target. For example, an array suitable comprises a feature selection (e.g., gene selection) such that each well of an array plate represents a target for analysis.

In embodiments, the qPCR arrays are designed for analysis of various biomarkers, including various nucleic acid molecules, for example, for analysis of messenger RNA (mRNA), for analysis of micro RNA (miRNA), for analysis of long non-coding RNA (IncRNA), etc as well as combinations thereof.

As described herein, in suitable embodiments the qPCR arrays comprise one or more, suitably two or more, three or more, four or more or five or more control features (i.e., genes) including, but not limited to: ACTB, B2M, GUSB, HPRT1, RPL13A, S100A6, TFRC, YWHAZ, CFL1, RPS13, TMED10, UBB, ATP5B, GAPDH, HMBS, HSPCB, RPLPO, SDHA, UBC, PPIA, FLOT2, TMBIM6, TBT1, MRPL19 and RPLP0. In suitable embodiments, the arrays comprise 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or all 25 of the control features described herein.

In further embodiments, additional control features (reference genes) can also be included in the qPCR arrays, including features from animals other than humans, including for example, mouse, rat, monkey, dog, etc. Such reference features can be selected by utilizing the various methods described herein applied to information from other animals.

Further exemplary reference features include, for example,

Mouse reference features;

| | |
|---|---|
| Actb | NM_007393 |
| B2m | NM_009735 |
| Gapdh | NM_008084 |
| Gusb | NM_010368 |
| Hsp90ab1 | NM_008302 |

Rat reference features:

| | |
|---|---|
| Actb | NM_031144 |
| B2m | NM_012512 |
| Hprt1 | NM_012583 |
| Ldba | NM_017025 |
| Rplp1 | NM_001007604 |

Cow reference features:

| | |
|---|---|
| ACTB | NM_173979 |
| GAPDH | NM_001034034 |
| HPRT1 | NM_001034035 |
| TBP | NM_001075742 |
| YWHAZ | NM_174814 |

Rhesus Macaque reference features:

| | |
|---|---|
| ACTB | NM_001033084 |
| B2M | NM_001047137 |
| GAPDH | XM_001105471 |
| LOC709186 | XM_001097691 |
| RPL13A | XM_001115079 |

miRNA reference features:

| | |
|---|---|
| SNORD61 | MS00033705 |
| SNORD68 | MS00033712 |
| SNORD72 | MS00033719 |
| SNORD95 | MS00013726 |
| SNORD96A | MS00033733 |
| RNU6-2 | MS00033740 |

In still further embodiments, the methods described herein provide methods of assigning a single probability score to one or more biomarkers. Suitably, such methods comprise collecting a sample set. Suitably, such sample sets are nucleic acid solutions, but can also be cell or tissue samples, blood samples, saliva samples, urine samples or other biological fluid samples, and can further comprise various proteins or other biological materials.

Suitably, nucleic acid molecules are extracted tram each sample of the sample set. Methods for carrying out such extraction are well known in the art.

Each nucleic acid molecule is then interrogated with the qPCR arrays as described herein. As used herein “interrogating” refers to applying the sample(s) to one or more locations (i.e., wells) of the array. The methods suitably comprise evaluating the discrimination power of one or more independent features. That is, the ability of one or snore features (e.g., genes) of the array is evaluated to determine how well they discriminate between a characteristic of biomarker (i.e., disease vs. non-disease state).

The methods further comprise generating a combined feature by analyzing the discrimination power of combinations of two or more independent features with one or more mathematical models. Methods for generating the combined feature, including the mathematical models utilized, are described herein and include for example, Random forest (RF) modeling, support vector machine (SVM) modeling and nearest shrunken centroid (NSC) modeling. Additional models known in the art can also be utilized in the methods described herein, including for example, various genetic algorithms, decision tress and Naïve Bayes modeling.

The methods then further comprise assigning a single probability score to the combined features. That is, a single value is assigned to the combined features that can be utilized to determine whether or not the level of a biomarker is indicative of the measured/desired outcome. The “cut-off” value for a biomarker—the probability score below or above which the presence of a biomarker is determinative—is suitably scalable, i.e., up or down as desired.

In exemplary embodiments, the interrogating comprises evaluating 2 to 40 independent features (i.e., genes) on a single array. As described herein, arrays are suitably 96 well plates, and thus the desired number of feature is suitably dependent upon the physical characteristics of the plates (number of wells in a row or column) and the ability to deposit the features (e.g., genes, etc.) on the plate. In suitable embodiments, the interrogating comprises evaluating 2 to 8 independent features, 8 to 16 independent features, 16 to 24 independent features, 24 to 32 independent features, 32 to 40 independent features, or 20 independent features, as well as values and ranges within these ranges.

The methods provided herein use microarray data for feature selection and then use selected targets to generate industry standard qPCR arrays with new clinical sample assay data in order to build a classification model. This multi-step method overcomes the disadvantages of traditional biomarker identification.

The methods provided herein use one microarray platform for feature selection analysis to avoid problems related to platform normalization and merging datasets.

The methods provided herein suitably use 7 target genes (much less than previous panels) together with controls to generate dCt data to input into machine learning model for classification. (Diagnosis).

Provided herein is a model-based classification system. After training and testing, the model is fined and only requires the input of new sample data to the model. The classification is calculated without the need of any old training data.

Provided herein is a model that uses tissue-specific input controls that can provide a more accurate comparison between samples, unlike the general microarray or qPCR controls that were traditionally used.

Provided herein, is a model that, even with a training set, achieves 88% accuracy and 82% specificity with 2-group K-means cluster analysis, 92% accuracy and 82% specificity with an unsupervised, hierarchical cluster analysis, and suitably classifies the training set 100% correctly.

The methods herein provide a practical molecular diagnostic qPCR assay signature panel based on machine learning classification models to identify malignant thyroid nodule.

In order to better distinguish malignant thyroid nodules from benign ones, the methods provided herein use a more practical qPCR platform. Thyroid cancer and control sample data set from microarray assay are used for final feature selection for thyroid malignancy identification. Several feature selection methods (such as Random Forest and Support Vector Machine) are used to rank the target. With the selected gene, a 384-well qPCR array (including 10 selected specific thyroid nodule housekeeping genes and 3 qPCR assay controls) are used to study a set of 49 benign and malignant thyroid samples for the signature panel development. Five housekeeping genes are further identified based on analysis. A fine toned classification signature (7 target genes and 5 controls) is developed using random forest classification model. Besides the training set, the methods provided herein also work, well on a test set that differing from the training set. The methods provide 91.7% accuracy, 87.5% sensitivity and 100% specificity, 100% PPV and 80% NPV. In a mixed sample test, the methods identify a tumor sample that only contains 25% real malignant samples mixed with 75% benign sample. These results suggest that the disclosed biomarker PCR array system is an efficient tool for biomarker development.

The methods provided herein focus on a panel of quantitative molecular classifiers that can distinguish, malignant thyroid nodules from benign or normal tissue. Provided is a method that uses a biomarker assay friendly platform-real-time PCR to achieve better accuracy, specificity and consistency for measuring the target nucleotide expression level tor the defined classification. Provided is a method that uses tissue-specific normalization control panels for better normalization of target gene expression and provides a solid base for biomarker use in clinical practice. Provided herein is a thyroid nodule malignancy biomarker generated through a cross validated and cross platform re-classified way. The biomarker comes from high-throughput screening feature selection-qPCR array development with control development-qPCR army sample assay and real-time PCR data analysis and classification signature re-identification. The results demonstrate strong performance in identification of malignant samples.

Provided is a biochemical gene expression classification system to classify thyroid nodules especially when standard pathology examination is ambiguous or indeterminate.

Thyroid tissue microarray gene expression data can be used with four machine learning-based gene ranking and selection methods: Random Forest (RF), Nearest Shrunken Centrokis (NSC), Bayesian factor Regression Modeling (BFRM) and Support Vector Machine (SVM). Previously identified target lists are also, used in the final target gene list.

Targets in the panel provided herein can also be replaced with other targets. Suitable replacements include:
NFC2 in the panel can be replaced with its highly correlated alternatives such, as RXRG, CITED1, TGFA, GALE, KLK10, LRP4, CDH3, NAB2, HMGA2, DPP4, SDC4, TIPARP, S100A11, PSD3, LGALS3, RAB27A, ADORA1, TACSTD2, KLK11, DUSP4, TIMP1, PIAS3, CTSH, MRC2, SCBL, ABCC3, CHBL1, TSC22D1, PROS1, QPCT, ODZ1, IGFPB6, RRAS, CAPN3, KRT19, SFN, ENDOD1, PLP2, PDLIM4, DOCK9, MAPK4, CDH16, KIT, MATN2, TLE1, ANK2, KIAA1467, COL9A3, TCFL5, TEAD4, SNTA1.
S100A11 1n the panel can be replaced with its highly correlated alternatives such as TIMP1, CH13L1, SFN, LGALS3, MRC2, MVP, NPC2, DPP4, CYP1B1, TACSTD2, PROS1, FN1, RXRG, PDLIM4, DUSP6, CTSH, ABCC3, MTMR11, SDC4, IGFBP6, PLAUR, PIAS3, TIPARP, RRAS, ANXA1, QPCT, MAPK4, KIT, TLE1, KIAA1467, SNTA1, SORBS2, GPR125.
SDC4 in the panel can be replaced with its highly correlated alternatives such as, TACSTD2, MET, PDLIM4, SERPINA1, TIPARP, TGFA, TSC22D1, GALE, LGALS3, NPC2, CYP1B1, FN1, IL1RAP, KLK10, ZNF217, DUSP5, CTSH ANXA1, CHI3L1, DPP4, MSN, RXRG, PROS1, SFN, BID, DUSP6, ENDOD1, DTX4, TIMP1, NRIP1, CD55, NAB2, PIAS3, S100A11, PRSS23, SCEL, LAMB3, CDH3, IGFBP6, CDC42EP1, HMGA2, ADORA1, SLC4A4, HGD, SORBS2, ELMO1, TFF3, TPO, KIT, ITPR1, MAPK4, FMOD, MT1F, FHL1, SLC39A14, TLE1, VEGFB, CDH16, SNTA1. ANK2.
CD53 in the panel can be replaced with its highly correlated alternatives such as, TMSB4X, SELL, CD86, CCR7, PLAUR, MYO7A, NFKBIE, S100B, and ARBGEF5.
MET in the panel can be replaced with its highly correlated alternatives such as, SDC4, TACSTD2, DTX4, IL1RAP, LGALS3, TGFA, GALE, KLK10, PARP4, HMGA2, PDLIM4, CHI3L1, SERPINA1, PROS1, TIPARP, FN1, ENDOD1, SLC39A14, HGD, ELMO1, TPO, SORBS2.
CHI3L1 in the panel can be replaced with its highly correlated alternative such as, LGALS3, TiMP1, DPP4, PDLIM4, SFN, CYPIB1, ENDOD1, KRT19, CTSH, TACSTD2, PROS1, ANXA1, PLAUR, S100A11, FN1, DUSP5, PLAU, SERPINA1, TIPARP, KLK10, S100B, MVP, IGF8P6, RAB27A, CDH3, SDC4, IL1RAP, MRC2, ABCC3, BID, NFC2, ADORA1, SLPI, LAMB3, RXRG, DUSP6, GALE, CITED1, TGFA, SCEL, RRAS, MET, ZFP36L1, CDS5, ZNF217, RIJNX1, SELL, PLP2, MYO7A, KIT, ELMO1, KIAA1467, TPO, SORBS2, HGD, CDH16, ADIPOR2, MATN2, SLC4A4, FASTK, MTIF, MAPK4, PRPS1, SNTA1, HMGCR, ITPR1, PGF, HK1, MPPED2, DIO1, TRAPFC6A, PRUNE, NDUFA2, FHL1, ARHGEF5, FLRT1, TFF3, CSRP2, SLC39A14, TLE1, TMEM50B, POLD2, FARS2, BMP7, BDH1, FCGBP, TCFL5, PEG3, GPR125, PGD, HSPB11, COL9A3, FKBP4, BCAT2.

TABLE 1
Thyroid nodule malignancy classification gene panel

Targets gene
NPC2, S100A11, SDC4, CD53, MET, GCSH, CHI3L1.
Reference genes
TBP, RPL13A, RPS13, HSP90AB1, YWHAZ.

The panel provided herein works well on a test set that is totally different from the training set. It can reach 91.7% accuracy, 87.5% sensitivity and 100% specificity, 100% PPV and 80% NPV. It also demonstrates its power In a mixed sample test, which can identify a tumor sample that only contains 25% real malignant samples and is mixed with 75% benign sample. These results suggest that the invented thyroid malignancy biomarker is an efficient tool for clinical diagnosis.

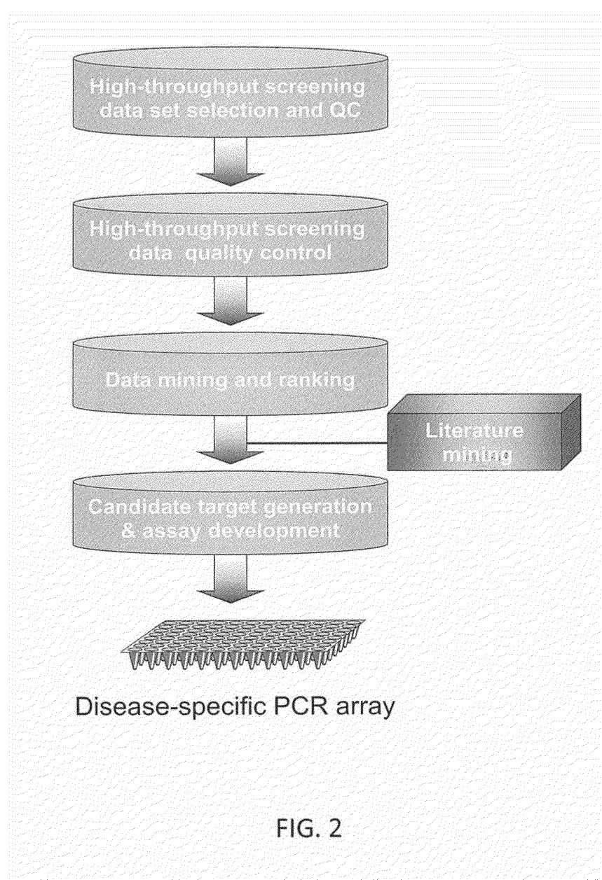

As shown in FIG. 2, in embodiments, high-throughput gene expression data sets are selected based on research interest, study objective, species and quality [minimum sample numbers, well-defined sampling conditions, availability of annotation, and uniformity of experimental data (signal intensity, outliers etc.)].

Selected data sets are normalized and then analyzed by multiple mathematical models including Random forest (RF), support vector machine (SVM) and nearest shrunken centroid (NSC). Top-ranked targets from all statistical analyzes and literature mining are combined to produce the final candidate gene list.

Quantitative real time PCR assays for all candidate genes are designed and tested for technical sensitivity, specificity, and dynamic range. Tissue-specific normalization control assays and performance controls are added to complete the final disease-specific qPCR array.

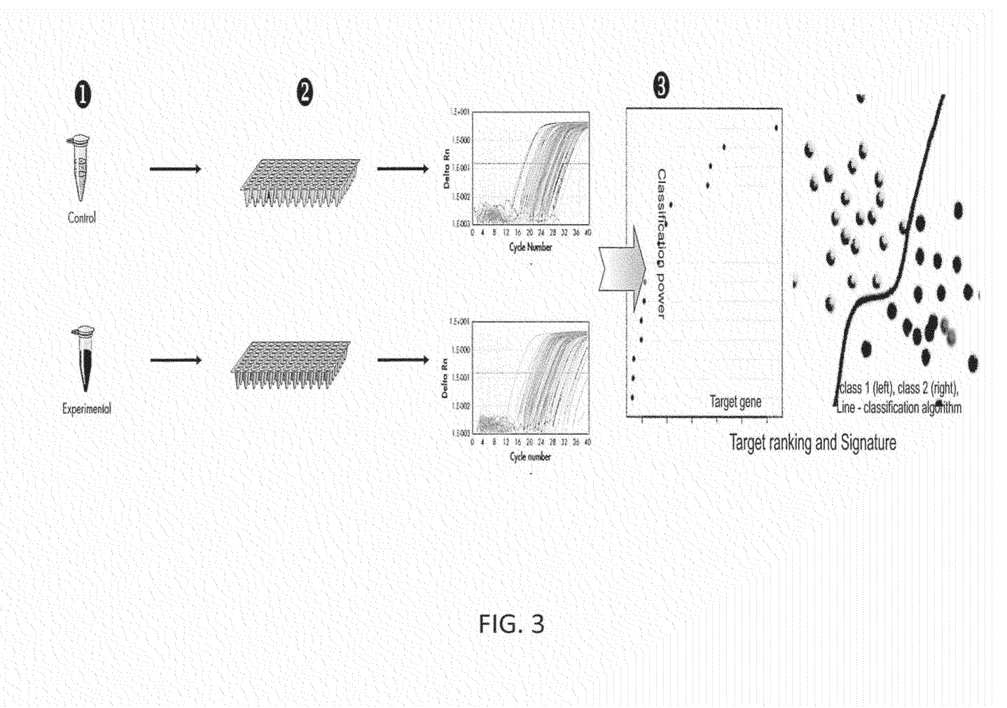

FIG. 3 shows a workflow from sample to biomarker signature panel using the disease-specific PCR array system. Researcher's efforts: 1) Sample collection and processing, then 2) qPCR is performed to get C_{T} values. 3) Shows Data analysis postal:

A. Normalization of gene expression, with final normalization gene panel selected based on expression stability of researcher's samples, to obtain ΔC₁.

B. Ranking of target genes for their classification power with RF ranking tool. Removal of unqualified targets (such as targets with no or low detection in both groups) for better assay stability.

C. Creation of a biomarker signature panel and classification algorithm using the RF model and cross validation.

qPCR Arrays for Thyroid Classification

In embodiments, quantitative real-time polymerase chain reaction (qPCR) arrays are provided. Suitably, the arrays comprise one or more thyroid nodule malignancy classification biomarkers. Suitable such biomarkers classification biomarkers are selected from the group of genes including, but not limited to, NPC2, S100A11, SDC4, CD53, MET, GCSH, and CHI3L1. The arrays further comprise one or more reference genes including, but not limited to, TBP, RPL3A, RPS13, HSP90AB1 and YWHAZ. The arrays further comprise a companion classifying algorithm for producing a single malignancy score and a. scalable cut-off threshold.

Exemplary algorithms and methods for producing such, algorithms, including the various mathematical models, are described herein.

As used herein, “malignancy score” refers to a single probability value or score assigned to a data set that is analyzed using the qPCR array.

As used heroin, a “cut-off threshold” refers to a low or high limit, depending oh the application, for a biomarker—the probability score below or above which the presence of a biomarker is determinative—is suitably scalable, i.e., up or down as desired. For example, in the case of malignancy classification, the cut-off threshold suitably delineates malignant from benign samples.

In embodiments, the qPCR arrays comprise 2 or more, 3 or more, 4 or more, 5 or more, 6 or more or all of the thyroid nodule malignancy classification biomarkers. In embodiments, the qPCR arrays comprise 2 or more, 3 or more, 4 or more or all of the reference genes. The qPCR arrays suitable comprise any combination of thyroid nodule malignancy classification biomarkers and reference (or control) genes.

Suitably the qPCR arrays comprise the thyroid nodule malignancy classification biomarkers NPC2, S100A11, SDC4, CD53, MET, GCSH, and CHI3L1 and the reference genes TBP, RPL13A, RPS13, HSP90AB1 and YWHAZ.

As described herein, the genes described for use in the qPCR arrays can be replaced by highly correlated alternative genes. For example, NPC2 in the arrays is replaced with a gene selected from the group consisting of RXRG, CITED1, TGFA, GALE, KLK10, LRP4, CDH3, NAB2, HMGA2, DPP4, SDC4, TIPARP, S100A11, PSD3, LGALS3, RAB27A, ADORA1, TACSTD2, KLK11, DUSP4, TIMP1, PIAS3, CTSH, MRC2, SCEL, ABCC3, CHI3L1, TSC22D1, PROS1, QPCT, ODZ1, IGFBP6, RRAS, CAPN3, KRT19, SFN, ENDOD1, PLP2, PDLIM4, DOCK9, MAPK4, CDH16, KIT, MATN2, TLE1, ANK2, KIAA1467, COL9A3, TCFL5, TEAD4 and SNTA1.

In embodiments, S100A11 in the arrays is replaced with a gene selected trout the group consisting of TIMP1, CHI3L1, SFN, LGALS3, MRC2, MVP, NPC2, DPP4, CYPIB1, TACSTD2, PROS1, FN1, RXRG, PDLIM4, DUSP6, CTSH, ABCC3, MTMR11, SDC4, IGFBP6, PLAUR, PIAS3, TIPARP, RRAS, ANXA1, QPCT, MAPK4, KIT, TLE1, KIAA1467, SNTA1, SORBS2 and GPR125.

In embodiments, SDC4 in the arrays is replaced with a gene selected from the group consisting of TACSTD2, MET, PDLIM4, SERPINA1, TIPARP, TGFA, TSC22D1, GAPE, LGALS3, NPC2, CYPIB1, FN1, IL1RAP, KLK10, ZNF217: DUSP5, CTSH, ANXA1, CHI3L1, DPP4, MSN, RXRG, PROS1, SFN, BID, DUSP6, ENDOD1, DTX4, TIMP1, NRIP1, CD55, NAB2, PIAS3, S100A11, PRSS23, SCEL, LAMB3, CDH3, IGFBP6, CDC42EP1, HMGA2, ADORA1, SLC4A4, HGD, SORBS2, ELMO1, TFF3, TPO, KIT, ITPR1, MAPK4, FMOD, MTIF, FHL1, SLC3PA14, TLE1, VEGFB, CDH16, SNTA1 and ANK2.

In embodiments, CDS53 in the array is replaced with a gene selected from the group consisting of TMSB4X, SELL, CD86, CCR7, PLAUR, MYO7A, NFKBIE, S100B, and ARHGEF5.

In embodiments, MET in the arrays is replaced with a gene selected from the group consisting of SDC4, TACSTD2, DTX4, IL1RAP, LGALS3, TGFA, GALE, KLK10, PARP4, HMGA2, PDLIM4, CHI3L1, SERPINA1, PROS1, TIPARP, FN1, ENDOD1, SLC39A14, HGD, ELMO1, TPO, SORBS2.

In embodiments, CHI3L1 in the arrays is replaced with a gene selected from the group consisting of LGALS3, TIMP1, DPP4, PDLIM4, SFN, CYPIB1, ENDOD1, KRT19, CTSH, TACSTD2, PROS1, ANXA1, PLAUR, S100A11, FN1,L DUSP5, PLAU, SERPINA1, TIPARP, KLK10, S100B, MVP, IGFBP6, RAB27A, CDH3, SDC4, IL1RAP, MRC2, ABCC3, BID, NPC2, ADORA1, SLP1, LAMB3, RXRG, DUSP6, GALE, CITED1, TGFA, SCEL, RRAS, MET, ZFP36L1, CD55, ZNF217, RUNX1, SELL, PLP2, MYO7A, KIT, ELMO1, KIAA1467, TPO, SORBS2, HGD, CDH16, ADIPOR2, MATN2, SLC4A4, FASTK, MTIF, MAPK4, PRPS1, SNTA1, HMGCR, ITPR1, PGF, HK1, MPPED2, DIO1, TRAPPC6A, PRUNE, NDUFA2, FHL1, ARHGEF5, FLRT1, TFF3, CSRP2, SLC39A14, TLE1, TMEM50B, POLD2, FARS2, BMP7, BDH1, FCGBP, TCFL5, PEG3, GPR125, FGD, HSPB11, COL9A3, FKBP4, BCAT2.

As described herein, the companion algorithm is based on Random forest (RF) modeling, or can be based on supporting vector machine (SVM) modeling, or can be based on Bayesian regression model (BRM) modeling, or any combination of these models.

It will be readily apparent to one of ordinary skill in the relevant arts that other suitable modifications and adaptations to the methods and applications described herein can be made without departing from the scope of any of the embodiments. It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

### EXAMPLES

### Example 1

### qPCR Method

Total RNA was reverse transcribed to complementary DNA (cDNA) according to the manufacturer's protocol (Qiagen, QuantiTECT reverse transcription kit, Valencia, Calif.). SYBR Green Biomarker Custom PCR arrays was used for gene expression detection. All the primers were synthesized by Integrated DNA Technologies (IDT, Coralville, Iowa). A quality control procedure was followed to ensure specificity and efficiency with a serial dilution of reference universal genomic DNA and cDNA. Amplification specificity was confirmed by agarose gel electrophoresis of the PCR products. Customized 384-well primer plates were printed. For each sample, cDNA equal to 0.8 ng total RNA input was mixed with SYBR Green master mix (QuantiTECT SYBR Green PCR Kit, Qiagen) in a 10 micro litter reaction volume. qPCR amplification was done on ABI 7900HT Real-time PCR System. Amplification was carried out for 40 cycles (at 94° C. for 15 seconds, at 55° C. for 30 seconds, and at 72° C. for 30 seconds). Dissociation curves generated at the end of each run were examined to verify specific PCR amplification, and absence of primer dimmer formation.

### Example 2

### Thyroid Malignancy qPCR Array

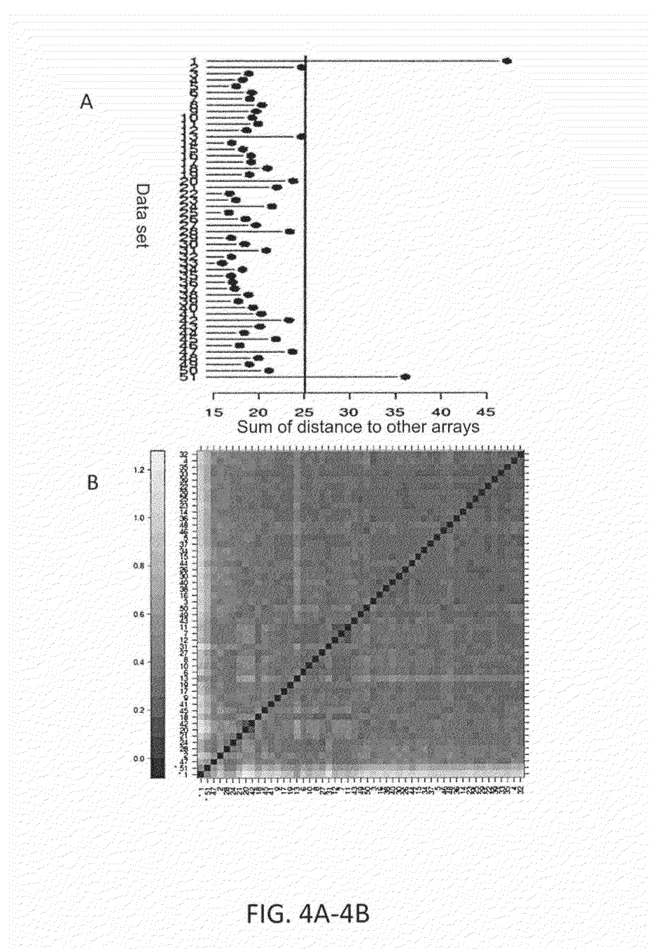

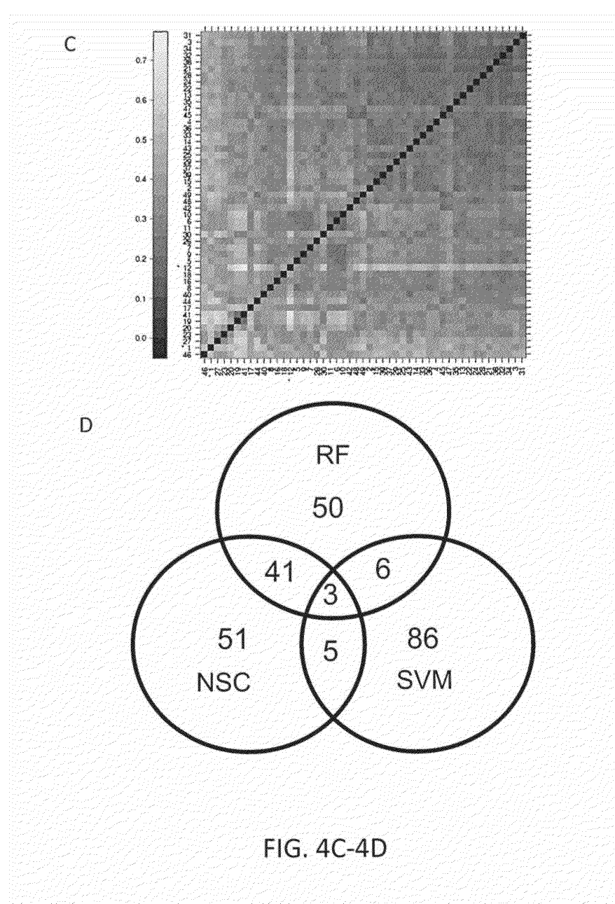

The published literature was searched and published high-throughput screening (microarray) data from 51 benign and malignant thyroid samples were selected for study. Outlier samples were identified and are shown in FIG. 4A. Outlier samples were removed from the dataset because they impaired sample clustering as shown in FIG. 4B. Sample clustering improved with removal of the outliers as shown in FIG. 4C. Multiple mathematical models including RF, NSC and SVM were used for biomarker candidate selection, and genes selected based on the literature were added for better potential biomarker coverage. FIG. 4D shows the overlap of the top 100 genes across the three representative mathematical models. qPCR assays were then performed on the top-ranked targets and were optimized tor their sensitivity, specificity and efficiency. Target assays meeting the QC standards were used for thyroid malignancy qPCR array. Ten normalization reference gene candidates were selected based on gene expression stability analysis with representative benign and malignant thyroid samples. Ultimately, 371 target assays, 10 normalization controls and 3 performance controls were used on a 384-well thyroid malignancy PCR array.

Forty-nine pathology-assessed thyroid, nodule samples (fresh frozen, 23 malignant and 26 benign, Weill Medical College of Cornell University) were tested using the thyroid malignancy PCR array. Normalization genes were selected based on gene expression stability and inter-group variation. The geometric mean of 5 selected normalization genes was used to normalize target gene expression. Normalized CT values were analyzed using an RF classification model. The optimization algorithm identified a panel of 12 genes as a gene expression signature for thyroid malignancy, shown below in Table 1.

TABLE 1
Thyroid Malignancy Gene Expression Signature

| | | | | | |
|---|---|---|---|---|---|
| NPC2 | S100A11 | SDC4 | CD53 | MET | GCSH |
| CHI3L1 | TBP | RPL13A | RPS13 | HSP90AB1 | YWHAZ |

Twelve pathology-assessed thyroid nodule samples (RNA from fresh frozen tissue; 8 malignant and 4 benign) were evaluated using the identified thyroid malignancy gene expression signature and a companion classification algorithm. Malignant thyroid nodule samples were successfully distinguished from benign nodules samples with 92% accuracy and 100% specificity in this limited size, independent dataset, as shown in Table 2.

TABLE 2
Prediction Results

| | Accuracy | Sensitivity | Specificity | PPV | |
|---|---|---|---|---|---|
| | (%) | (%) | (%) | (%) | NPV (%) |
| Prediction | 91.7 | 87.5 | 100.0 | 100.0 | 80.0 |
| result | | | | | |

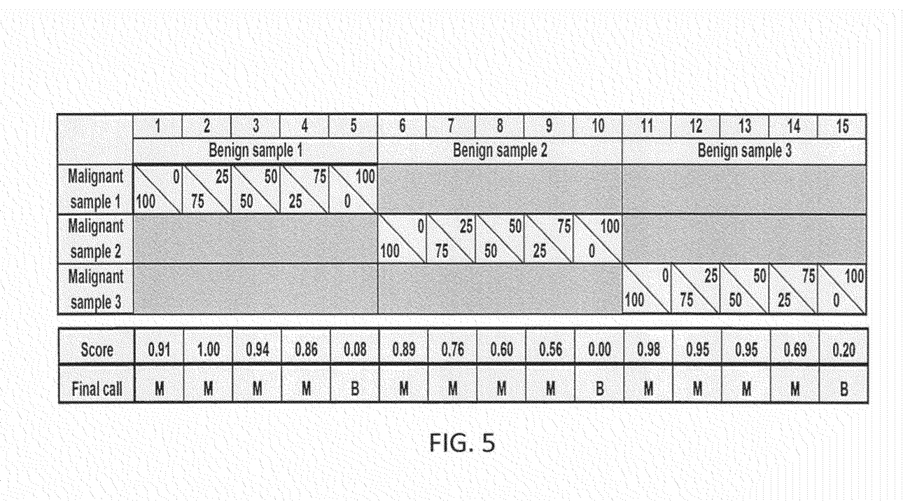

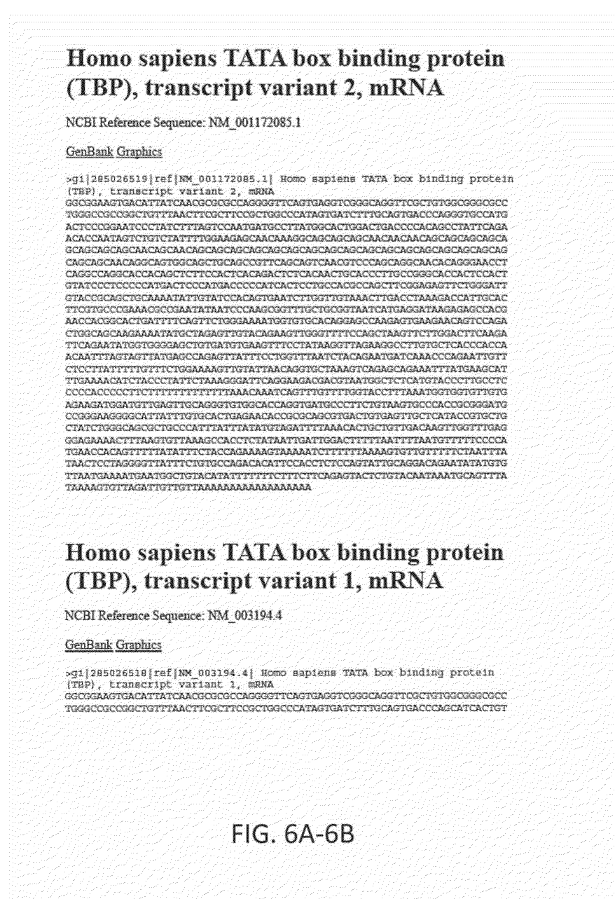

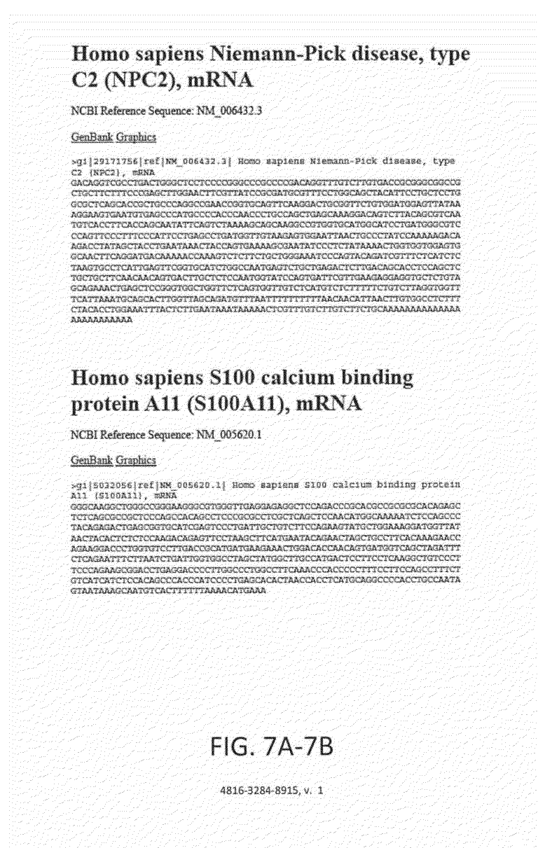

Three pairs of benign and malignant thyroid samples were mixed in different ratios and analyzed using the thyroid malignancy gene expression signature and companion classification algorithm. Analysis results provided a malignancy score for each sample and distinguished mixed samples containing as little as 25% malignant sample from pure benign samples with 100% accuracy, as shown in FIG. 5. Malignant-Scored>0.5 (M), Benign-Score<0.5 (B).

### Example 3

### Additional Panel Development

A 20 reference gene panel was tested (data not shown) with 6 thyroid samples covering normal and different stage of thyroid tumor (OriGene, Rockville, Md.). The top 10 genes were selected based on their expression stability and variation between benign and cancer group. When the final qPCR results were collected with all thyroid samples, reference gene expression was further analyzed. The reference genes with the smallest difference between benign and malignant groups and highest expression stability were picked. Five genes were selected as reference genes; TBP, RPL13A, RPS13, HSP90AB1 and YWHAZ.

A repetitive gene selection and ranking process was then repeated with random forest (RF). Target genes were pre-filtered with their expression level and the relative expression: range difference. The genes with no or extremely low expression, as well as the gene that have limited difference (<0.5 ΔCt, easily to be reversed by qPCR variation), were removed from the full list. A final list of 189 genes was used to rank their importance based on their classification power in a Random Forest model system. The area under Receiver Operating Characteristics curve (AUC) was evaluated with bootstrap methods.

Finally a thyroid nodule malignancy classification biomarker was identified in a panel of real-time PCR assay targets NPC2, S100A11, SDC4, CD53, MET, GCSH, and CHI3L1. The normalized expression levels were determined using the delta-delta Ct method with a panel of reference genes consisting of TBP, RPL13A, RPS13, HSP90AB1 and YWHAZ.

The performance of the trained RF classification model is also tested with 12 thyroid tissue samples and 20 artificial mixed samples.

TABLE 3

| Position | Gene Symbol |
|---|---|
| A1 | ABCC3 |
| A2 | ANK2 |
| A3 | ACTR3 |
| A4 | ANXA1 |
| A5 | ACVR1B |
| A6 | ANXA2P1 |
| A7 | ADCY7 |
| A8 | ANXA6 |
| A9 | ADH5 |
| A10 | AP2B1 |
| A11 | ADPOR2 |
| A12 | AP2B1 |
| A13 | ACORA1 |
| A14 | APOBEC3B |
| A15 | AHCYL2 |
| A16 | ARHGAP5 |
| A17 | ARNAK |
| A18 | ARHBEF5 |
| A19 | AIM1 |
| A20 | ARL2 |
| A21 | AIMP2 |
| A22 | ATOX1 |
| A23 | ALDOA |
| A24 | ATP5H |
| B1 | MET2 |
| B2 | MTMR11 |
| B3 | MF367 |
| B4 | MTMR4 |
| B5 | MFRN2 |
| B6 | MTU31 |
| B7 | MUL4 |
| B8 | MTX1 |
| B9 | MMP11 |
| B10 | MUC1 |
| B11 | MPPED2 |
| B12 | MVP |
| B13 | MRC2 |
| B14 | MYH10 |
| B15 | MRPL12 |
| B16 | MYO7A |
| B17 | MSN |
| B18 | NAB2 |
| B19 | MT1F |
| B20 | NCAM1 |
| B21 | MT1G |
| B22 | NCRNA00004 |
| B23 | MTCP1NB |
| B24 | NDUFA2 |
| C1 | ATP512 |
| C2 | BRCA2 |
| C3 | ATP5S |

It will be readily apparent to one of ordinary skill an the relevant arts that other suitable modifications and adaptations to the methods and applications described herein can be made without departing from the scope of any of the embodiments.

It is to be understood that while certain embodiments have been illustrated and described herein, the claim are not to be limited to the specific tonus or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

All publications, patents and patent applications mentioned in this specification are herein incorporated by reference to the same extent as if each individual publication, patent or patent application was specifically and individually indicated to be incorporated by reference.

1. A quantitative real-time polymerase chain reaction (qPCR) array comprising:
a. one or more thyroid nodule malignancy classification biomarkers selected from the group consisting of NPC2, S100A11, SDC4, CD53, MET, GCSH, and CHI3L1;
b. one or more reference genes selected from the group consisting of TBP, RPL13A, RPS13, HSP90AB1 and YWHAZ; and
c. a companion classifying algorithm for producing a single malignancy score and a scalable cut-off threshold.

2. The qPCR array of claim 1, comprising 3 or more of the thyroid nodule malignancy classification biomarkers and 3 or more of the reference genes.

3. The qPCR array of claim 1, comprising 5 or more of the thyroid nodule malignancy classification biomarkers and 4 or more of the reference genes.

4. The qPCR array of claim 1, comprising the thyroid nodule malignancy classification biomarkers NPC2, S100A11, SDC4, CD53, MET, GCSH, and CHI3L1 and the reference genes TBP, RPL13A, RPS13, HSP90AB1 and YWHAZ.

5. The qPCR array of claim 1, wherein NPC2 in the array is replaced, with a gene selected from the group consisting of RXRG, CITED1, TGFA, GALE, KLK10, LRP4, CDH3, NAB2, HMGA2, DPP4, SDC4, TIPARP, S100A11, PSD3, LGALS3, RAB27A, ADORA1, TACSTD2, KLK11, DUSP4, TIMP1, PIAS3, CTSH, MRC2, SCEL, ABCC3, CHI3L1, TSC22D1, PROS1, QPCT, ODZ1, IGFBP6, RRAS, CAPN3, KRT19, SFN, ENDOD1, PLP2, PDLIM4, DOCK9, MAPK4, CDH16, KIT, MATN2, TLE1, ANK2, KIAA1467, COL9A3, TCFL5, TEAD4 and SNTA1.

6. The qPCR array of claim 1, wherein S100A11 in the array is replaced with a gene selected from the group consisting of TIMP1, CHI3L1, SFN, LGALS3, MRC2, MVP, NPC2, DPP4, CYPIB1, TACSTD2, PROS1, FN1, RXRG, PDLIM4, DUSP6, CTSH, ABCC3, MTMR11, SDC4, IGFBP6, PLAUR, PIAS3, TIPARP, RRAS, ANXA1, QPCT, MAPK4, KIT, TLE1, KIAA1467, SNTA1, SORBS2 and GPR125.

7. The qPCR array of claim 1, wherein SDC4 in the array is replaced with a gene selected from the group consisting of TACSTD2, MET, PDLIM4, SERPINA1, TIPARP, TGFA, TSC22D1, GALE, LGALS3, NPC2, CYP1B1, FN1, IL1RAP, KLK10, ZNF217, DUSP5, CTSH, ANXA1, CHI3L1, DPP4, MSN, RXRG, PROS1, SFN, BID, DUSP6, ENDOD1, DTX4, TIMP1, NRIP1, CD55, NAB2, PIAS3, S100A11, PRSS23, SCEL, LAMB3, CDH3, IGFBP6, CDC42EP1, HMGA2, ADORA1, SLC4A4, HGD, SORBS2, ELMO1, TFF3, TPO, KIT, ITPR1, MAPK4, FMOD, MTIF, FHL1, SLC33A14, TLE1, VEGFB, CDH16, SNTA1, and ANK2.

8. The qPCR array of claim 1, wherein CD53 in the array is replaced with a gene selected from the group consisting of TMSB4X, SELL, CD86, CCR7, PLAUR, MYO7A, NFKBIE, S100B, and ARHGEF5.

9. The qPCR array of claim 1, wherein MET in the array is replaced with a gene selected from the group consisting of SDC4, TACSTD2, DTX4, IL1RAP, LGALS3, TGFA, GALE, KLK10, PARP4, HMGA2, PDLIM4, CHI3L1, SERPINA1, PROS1, TIPARP, FN1, ENDOD1, SLC39A14, HGD, ELMO1, TPO, SORBS2.

10. The qPCR array of claim , wherein CHI3L1 in the array is replaced with a gene selected from the group consisting of LGALS3, TIMP1, DPP4, PDLIM4, SFN, CYPIB1, ENDOD1, KRT19, CTSH, TACSTD2, PROS1, ANXA1, PLAUR, S100A11, FN1, DUSP5, PLAU, SERPINA1, TIPARP, KLK10, S100B, MVP, IGFBP6, RAB27A, CDH3, SDC4, IL1RAP, MRC2, ABCC3, BID, NPC2, ADORA1, SLPI, LAMB3, RXRG, DUSP6, GALE, CITED1, TGFA, SCEL, RRAS, MET, ZFP36L1, CD55, ZNF217, RUNX1, SELL, PLP2, MYO7A, KIT, ELMO1, KIAA1467, TPO, SORBS2, HGD, CDH16, ADIPOR2, MATN2, SLC4A4, FASTK, MTIF, MAPK4, PRPS1, SNTA1, HMGCR, ITPR1, PGF, HK1, MPPED2, DIO1, TRAPPC6A, PRUNE, NDUFA2, FHL1, ARHGEF5, FLRT1, TFF3, CSRP2, SLC39A14, TLE1, TMEM50B, POLD2, FARS2, BMP7, BDH1, FCGBP, TCFL5, PEG3, GPR125, PGD, HSPB11, COL9A3, FKBP4, BCAT2.

11. The qPCR array of claim 1, wherein the companion algorithm is based on random forest (RF) modeling.

12. The qPCR array of claim 1, wherein the companion algorithm is based on supporting vector machine (SVM) modeling.

13. The qPCR array of claim 1, wherein the companion algorithm is based on Bayesian Regression Model (BRM) modeling.


SEQUENCE LISTING (4 sequences)

SEQ ID NO: 1 - DNA, 1719 bp, Homo sapiens
ggcggaagtgacattatcaacgcgcgccaggggttcagtgaggtcgggcaggttcgctgtggcgggcgcctgggccgccggctgtttaacttcgcttccgctggcccatagtgatctttgcagtgacccagggtgccatgactcccggaatccctatctttagtccaatgatgccttatggcactggactgaccccacagcctattcagaacaccaatagtctgtctattttggaagagcaacaaaggcagcagcagcaacaacaacagcagcagcagcagcagcagcagcaacagcaacagcagcagcagcagcagcagcagcagcagcagcagcagcagcagcagcagcagcagcaacaggcagtggcagctgcagccgttcagcagtcaacgtcccagcaggcaacacagggaacctcaggccaggcaccacagctcttccactcacagactctcacaactgcacccttgccgggcaccactccactgtatccctcccccatgactcccatgacccccatcactcctgccacgccagcttcggagagttctgggattgtaccgcagctgcaaaatattgtatccacagtgaatcttggttgtaaacttgacctaaagaccattgcacttcgtgcccgaaacgccgaatataatcccaagcggtttgctgcggtaatcatgaggataagagagccacgaaccacggcactgattttcagttctgggaaaatggtgtgcacaggagccaagagtgaagaacagtccagactggcagcaagaaaatatgctagagttgtacagaagttgggttttccagctaagttcttggacttcaagattcagaatatggtggggagctgtgatgtgaagtttcctataaggttagaaggccttgtgctcacccaccaacaatttagtagttatgagccagagttatttcctggtttaatctacagaatgatcaaacccagaattgttctccttatttttgtttctggaaaagttgtattaacaggtgctaaagtcagagcagaaatttatgaagcatttgaaaacatctaccctattctaaagggattcaggaagacgacgtaatggctctcatgtacccttgcctcccccacccccttctttttttttttttaaacaaatcagtttgttttggtacctttaaatggtggtgttgtgagaagatggatgttgagttgcagggtgtggcaccaggtgatgcccttctgtaagtgcccaccgcgggatgccgggaaggggcattatttgtgcactgagaacaccgcgcagcgtgactgtgagttgctcataccgtgctgctatctgggcagcgctgcccatttatttatatgtagattttaaacactgctgttgacaagttggtttgagggagaaaactttaagtgttaaagccacctctataattgattggactttttaattttaatgtttttccccatgaaccacagtttttatatttctaccagaaaagtaaaaatcttttttaaaagtgttgtttttctaatttataactcctaggggttatttctgtgccagacacattccacctctccagtattgcaggacagaatatatgtgttaatgaaaatgaatggctgtacatatttttttctttcttcagagtactctgtacaataaatgcagtttataaaagtgttagattgttgttaaaaaaaaaaaaaaaaaa

SEQ ID NO: 2 - DNA, 140 bp, Homo sapiens
ggcggaagtgacattatcaacgcgcgccaggggttcagtgaggtcgggcaggttcgctgtggcgggcgcctgggccgccggctgtttaacttcgcttccgctggcccatagtgatctttgcagtgacccagcatcactgt

SEQ ID NO: 3 - DNA, 921 bp, Homo sapiens
gacaggtcgcctgactgggctcctccccgggcccgccccgacaggtttgtcttgtgaccgcgggcggccgctgcttctttcccgagcttggaacttcgttatccgcgatgcgtttcctggcagctacattcctgctcctggcgctcagcaccgctgcccaggccgaaccggtgcagttcaaggactgcggttctgtggatggagttataaaggaagtgaatgtgagcccatgccccacccaaccctgccagctgagcaaaggacagtcttacagcgtcaatgtcaccttcaccagcaatattcagtctaaaagcagcaaggccgtggtgcatggcatcctgatgggcgtcccagttccctttcccattcctgagcctgatggttgtaagagtggaattaactgccctatccaaaaagacaagacctatagctacctgaataaactaccagtgaaaagcgaatatccctctataaaactggtggtggagtggcaacttcaggatgacaaaaaccaaagtctcttctgctgggaaatcccagtacagatcgtttctcatctctaagtgcctcattgagttcggtgcatctggccaatgagtctgctgagactcttgacagcacctccagctctgctgcttcaacaacagtgacttgctctccaatggtatccagtgattcgttgaagaggaggtgctctgtagcagaaactgagctccgggtggctggttctcagtggttgtctcatgtctctttttctgtcttaggtggtttcattaaatgcagcacttggttagcagatgtttaattttttttttaacaacattaacttgtggcctctttctacacctggaaatttactcttgaataaataaaaactcgtttgtcttgtcttctgcaaaaaaaaaaaaaaaaaaaaaaaaa

SEQ ID NO: 4 - DNA, 595 bp, Homo sapiens
gggcaaggctgggccgggaagggcgtgggttgaggagaggctccagacccgcacgccgcgcgcacagagctctcagcgccgctcccagccacagcctcccgcgcctcgctcagctccaacatggcaaaaatctccagccctacagagactgagcggtgcatcgagtccctgattgctgtcttccagaagtatgctggaaaggatggttataactacactctctccaagacagagttcctaagcttcatgaatacagaactagctgccttcacaaagaaccagaaggaccctggtgtccttgaccgcatgatgaagaaactggacaccaacagtgatggtcagctagatttctcagaatttcttaatctgattggtggcctagctatggcttgccatgactccttcctcaaggctgtcccttcccagaagcggacctgaggaccccttggccctggccttcaaacccaccccctttccttccagcctttctgtcatcatctccacagcccacccatcccctgagcacactaaccacctcatgcaggccccacctgccaatagtaataaagcaatgtcacttttttaaaacatgaaa